(19) United States
(12) Patent Application Publication
(10) Pub. No.: US 2020/0335184 A1
(43) Pub. Date: Oct. 22, 2020

(54) TRACKING CLINICAL SAMPLES AND TRIALS WITH DISTRIBUTED LEDGER

(71) Applicant: HSBlox, Inc., Alpharetta, GA (US)

(72) Inventors: Rahul Sharma, Cumming, GA (US); Lynn Eliot Carroll, Atlanta, GA (US); Deepti Sharma, Cumming, GA (US); Apoorva Patel, Milton, GA (US); Johm Kenneth Layne, Marietta, GA (US)

(73) Assignee: HSBlox, Inc., Alpharetta, GA (US)

(21) Appl. No.: 16/851,813

(22) Filed: Apr. 17, 2020

Related U.S. Application Data
(60) Provisional application No. 62/836,289, filed on Apr. 19, 2019.

(51) Int. Cl. G16H 10/20; G06Q 20/40; G06Q 20/02; G06Q 50/22; H04L 9/06

(52) U.S. Cl. CPC G16H 10/20; G06Q 20/405; G06Q 20/02; G06Q 20/401; G06Q 2220/00; H04L 9/0643; H04L 2209/38; H04L 2209/56; G06Q 50/22

(57) ABSTRACT

A system and method for tracking clinical samples and clinical trials. The system includes a distributed ledger for enforcing one or more smart contracts, a plurality of entities joined to the distributed ledger, a plurality of nodes corresponding to the plurality of entities, and a management application. The plurality of entities include a trial sponsor entity, a trial site entity, a laboratory entity, and a research organization entity. Each entity of the plurality of entities comprises a corresponding enterprise system. The plurality of nodes corresponding to the plurality of entities. A corresponding node of the plurality of nodes communicates with the corresponding enterprise system of the plurality of entities using an applications programming interface (API) to communicate with the distributed ledger. The plurality of entities communicate using the API to engage in one or more smart contracts managed by the distributed ledger and related to the clinical samples and the clinical trials. The digital sample manager application provides a trusted user interface into the status of the one or more smart contracts.

### CROSS-REFERENCE TO RELATED APPLICATION(S)

This application claims priority to U.S. Provisional Patent Application Ser. No. 62/836,289, filed on Apr. 19, 2019, which incorporated herein by reference in its entirety.

### TECHNICAL FIELD

The present disclosure relates to systems for managing and tracking samples, such as clinical samples for clinical trials, using a distributed ledger. For example, digital ledger may be implemented using blockchain or related techniques.

### BACKGROUND

Generally stated, the success and prosperity of a pharmaceutical company can be measured by the breadth and quality of its pipeline coupled with its ability to maximize the value of each opportunity. In today's environment, with increasingly specialized and scarce opportunities to pursue, a complex web of local and international regulatory hoops to navigate, and unforgiving shareholder and market scrutiny, it is paramount to bring products to market quickly and reliably.

For example, from a clinical trials perspective, the quest for efficiency and specialization has driven pharmaceutical companies (Pharma) to increasingly seek the expertise of clinical research organizations (CROs), who carry the responsibility of meeting timelines, as well as quality assurance and data control requirements. CROs in turn look to sample laboratories, logistics partners, and specialized healthcare professionals to collect, store, and manage samples and data.

Another challenge addressed by the present disclosure arises as CROs and Pharma increasingly expand outside of their domains and beyond the developed world to generate a diverse and cost-effective network of trial sites and sources of patients. In such a case, the difficulty of achieving milestones and preserving acceptable data quality is magnified. For example, a recent study of Clinical Operations leaders from global life sciences companies showed that 80% of respondents stated they regularly miss trial milestones.

When conducting a pharma trial, a complex network of local and remote systems and data sources is assembled to support a typical trial. Trial and sample data may reside in disparate systems and are sourced from variety of core labs or ancillary vendors. As a result, 65% of Clinical Operations leaders report relying on outdated and manually compiled spreadsheets of data derived from multiple CTMS and EDC systems.

Disadvantageously, this data complexity leads to uncertainty for decision makers and several limitations. For instance, issues cannot be investigated in real-time because decision makers do not know or have visibility into all actions being taken to address issues. In another example, too much data can obfuscate the issues. As a result, the study metrics may be out of date, or may not be trusted by key stakeholders.

Therefore, current conditions are ripe for a new technology that will provide more visibility and access to data in real-time for Pharma Sponsors, CROs and other stakeholders.

### SUMMARY

In one aspect, a system for tracking clinical samples and clinical trials is provided. For instance, the system includes a distributed ledger for enforcing one or more smart contracts, a plurality of entities joined to the distributed ledger, a plurality of nodes corresponding to the plurality of entities, and a management application. The plurality of entities include a trial sponsor entity, a trial site entity, a laboratory entity, and a research organization entity. Each entity of the plurality of entities comprises a corresponding enterprise system. The plurality of nodes correspond to the plurality of entities. A corresponding node of the plurality of nodes communicates with the corresponding enterprise system of the plurality of entities using an applications programming interface (API) to communicate with the distributed ledger. The plurality of entities communicate using the API to engage in one or more smart contracts managed by the distributed ledger and related to the clinical samples and the clinical trials. The digital sample manager application provides a trusted user interface into the status of the one or more smart contracts.

In another aspect, a method for tracking clinical samples and clinical trials is presented. The method includes the following: Providing a distributed ledger for enforcing one or more smart contracts. Joining a plurality of entities to the distributed ledger, the plurality of entities comprising a trial sponsor entity, a trial site entity, a laboratory entity, and a research organization entity. Communicating between the plurality of entities and the distributed ledger using an application programming interface. Engaging at least two of the plurality of entities in one or more smart contracts managed by the distributed ledger and related to the clinical samples and the clinical trials. Providing a trusted user interface into the status of the one or more smart contracts

### BRIEF DESCRIPTION OF THE SEVERAL VIEWS OF THE DRAWINGS

A more particular description of the invention briefly summarized above may be had by reference to the embodiments, some of which are illustrated in the accompanying drawings. It is to be noted, however, that the appended drawings illustrate only typical embodiments of this invention and are therefore not to be considered limiting of its scope, for the invention may admit to other equally effective embodiments. Thus, for further understanding of the nature and objects of the invention, references can be made to the following detailed description, read in connection with the drawings in which:

FIG. 1 is a high-level diagram of multiple parties or entities engaged in a process to track clinical samples and/or trials, in accordance with one or more aspects of the present disclosure;

FIG. 2 is a block diagram of a system for tracking clinical samples and/or trials, in accordance with one or more aspects of the present disclosure;

FIG. 3 is a graphical user interface for tracking clinical samples and/or trials, in accordance with one or more aspects of the present disclosure;

FIGS. 4A & 4B are block diagrams providing further details of a system for tracking clinical samples and/or trials, in accordance with one or more aspects of the present disclosure; and

FIG. 5 is a flow chart of an exemplary method of tracking clinical samples and/or trials, in accordance with one or more aspects of the present technique.

### DETAILED DESCRIPTION

In the following description, some aspects will be described in terms that would ordinarily be implemented as software programs. Those skilled in the art will readily recognize that the equivalent of such software can also be constructed in hardware, firmware, or micro-code. Because data-manipulation algorithms and systems are well known, the present description will be directed in particular to algorithms and systems forming part of, or cooperating more directly with, systems and methods described herein. Other aspects of such algorithms and systems, and hardware or software for producing and otherwise processing the signals involved therewith, not specifically shown or described herein, are selected from such systems, algorithms, components, and elements known in the art. Given the systems and methods as described herein, software not specifically shown, suggested, or described herein that is useful for implementation of any aspect is conventional and within the ordinary skill in such arts.

The present disclosure relates to systems and methods for tracking, tracing, monitoring, or managing, e.g., clinical samples in clinical trials. Such a system can include a track and trace application for clinical samples, and may be referred to as a Digital Sample Manager (DSM) system, a system for managing clinical samples, or other similar names. The core functionality of such a system is to solve the logistics issues in the digital supply chain in healthcare.

Some conventional systems make use of a single “trusted” entity to control all of the data in the system. The problem with this model is that it requires that all participants have trust in that one entity, which may not be the case when many different entities from industry, governments, and research organizations are involved. Another disadvantage of conventional systems is that data can be out of date or stale, because the data is stored in systems that are not connected.

Advantageously, the systems and methods described herein allow multiple participants to remove the need for intermediaries, since transactions are verified, recorded and coordinated autonomously without a need for third party interactions. Another advantage of the present disclosure is to use distributed ledger and blockchain technology to overcome some of the limitations noted above caused by disparate data systems. Some of the features of the present disclosure, which is based on distributed ledgers, are now described.

One specific use case of the technique is in clinical samples tracking and tracing, where biological samples are tracked from an investigator site all the way to storage. Other embodiments of the technique are used to track and trace other physical or virtual objects, and are contemplated within the scope of the present disclosure. For instance, exemplary use cases exist for track and trace initiatives in pharmaceutical (Pharma) and Life Sciences applications, including use cases in the areas of drug development, supply chain, and channel distribution.

Next, certain aspects of terminology shall be discussed as follows:

Chain of Custody refers to knowing who has what, when and where. It is a chronological documentation of all parties that come in to contact with an item, and a history of all transactions.

Consensus Mechanism is a set of rules that validates a transaction and records it on the ledger as an immutable record.

Immutability means that once a record is written to the ledger, it cannot be altered. Participants to the transaction have electronically signed the record, confirming its validity.

Node(s) include the stakeholder entities participating in a distributed ledger transaction network of a system according to the present disclosure.

Notary is a distributed ledger network service that provides uniqueness consensus by attesting that, for a given transaction, it has not already signed other transactions. Notarization is the point of finality. This concept may be implemented in any suitable ledger system, such as the CORDA platform from R3, and similar approaches exist in other Distributed Ledgers like Hyperledger Fabric.

Data provenance is a historical record for any piece of data—where the data originated, tracking of changes made to the data, who made the changes, and who the data has been shared with over time. This provides assurance the information can be trusted for data validation and audit purposes.

A Smart Contract is computer code by which system participants agree to share information with each other. The smart contract is automatically enforced on the network when pre-defined rules are met. Smart contracts enforce business rules, enabling automation of processes, data sharing, and transaction processing. In one example, a smart contract is a computer protocol intended to digitally facilitate, verify, or enforce the negotiation or performance of a contract. Smart contracts allow the performance of credible transactions without third parties. These transactions are trackable and irreversible, using, e.g., a distributed ledger.

In one embodiment, a distributed ledger uses a blockchain to store and validate smart contract information. However, in other embodiments, the distributed ledger may use other data structures that are not chain based to store the data. Other properties that are inherent in blockchain and which help implement the blockchain include, but are not limited to, an immutable ledger, smart contracts, security, privacy, decentralization, consensus, endorsement, accessibility, etc. The smart contract aspect of a blockchain may use assets recorded on the distributed ledger to execute transactions and generate data values. Smart contracts may invoke changes to the distributed ledger.

Clinical Trial Management System (CTMS) is a software system used by biotechnology and pharmaceutical industries to manage clinical trials in clinical research. The system maintains and manages planning, performing and reporting functions, along with participant contact information, tracking deadlines and milestones.

Electronic Data Capture (EDC) system is a computerized system designed for the collection of clinical data in electronic format for use mainly in human clinical trials.

Sponsor is an entity that is paying for and/or conducting a clinical study for a new drug approval etc. This will be an example of companies like Sanofi, Eli Lilly, Merck etc. One sponsor can have multiple studies.

Study is a program that the Sponsor has created or contracted with the clinical research organization (CRO) to conduct and run tests. This is the highest level of tracking via Digital Manager Solution. One study will have multiple Clinical trial sites, multiple accessions, and multiple lab results.

Accession Number is the unique number for a given sponsor-study-sample kit-site combination that is for a visit at the clinical trial site. So, an Accession ID will have multiple samples included. Relationship is that one study can have multiple accession numbers (per visit). Each visit will have one accession number. Accession ID is for the kit which has multiple tubes (sample IDs).

Sample ID is the unique ID for each sample that is collected within a kit (accession number). This will be the granular level information tracked via DSM. There will be multiple samples within a kit and for an accession number- there will be multiple sample ID(s). Relationship is one accession has multiple sample ID(s).

Trial Site is the organization (provider practice, hospital, research institute) that is participating with the Sponsor to identify potential patient(s) (de-identified) for a given study. This will be the site where the samples will be collected and shipped from. Airbill scan will happen at this facility via mobile app. There can be one or multiple trial sites for one study.

Aliquot is the term used to describe a child sample that is created from a parent sample. There are scenarios where based on the results, additional testing will be needed and in that scenario, there are child samples created from the parent sample. These are called Aliquots. Relationship will be one sample can have one aliquot or multiple aliquots. These will be tracked via the sample ID (example 01 or 02 assigned to the parent sample ID).

The past few years have seen a tremendous surge in dialogue about blockchain and its potential to disrupt the healthcare and life sciences ecosystems. With multiple participating stakeholders and disparate systems, sharing data, validating its provenance, and addressing privacy concerns presents many challenges. Blockchain technology is showing promise in addressing the challenges, generating auditability and traceability between stakeholders with a single source of truth that reduces errors and the need for reconciliation. In simple terms, blockchain technology enables the participants who are privy to the shared facts of a transaction to know with 100% certainty that what one entity sees is what the others also see. Thus, this advantageously enables the participants in the distributed de-centralized network to trust the data even though these participants may not trust each other. However, conventional blockchain techniques have focused on financial transactions only. Disadvantageously, conventional systems have not addressed more complex situations in which multiple parties choose to work together in an eco-system. Advantageously, the present disclosure presents a technique in which several stakeholders can work together cooperatively yet at the same time can ensure that information is tracked and managed with absolute accuracy.

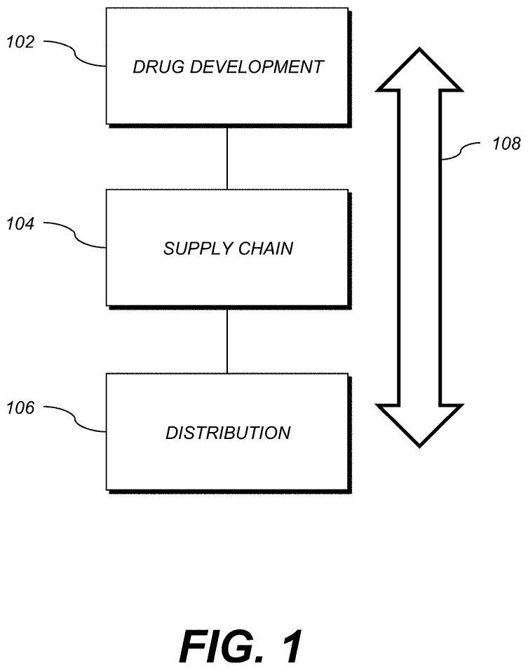

By way of overview, FIG. 1 depicts a high-level diagram of one embodiment of the applicability of a process 108 to multiple parties in, for example, the pharma industry, and specifically for clinical trials processes. In one example, the process 108 may be employed by a drug development entity 102. In another example, the process 108 may be employed by one or more supply chain entities 104. In a further example, the process 108 may be employed by one or more distribution channel entities 106. In one embodiment, the process 108 use a digital ledger based on blockchain technologies for the tracking and tracing of clinical samples in a clinical trials use case. As an advantage, the present system allows these entities 102, 104, 106 to participate in the process 108 with certainty that key process steps are validated.

Other example stakeholders, across multiple use cases, may include: Pharmaceutical Manufacturers, Distributors, Trial or Research Sponsors, Clinical Trial Sites, Labs, Clinical Research Organizations, Food and Drug Administration (FDA) and Internal Auditors, Shippers, Local and Third-Party Labs, or Patients.

With respect to the entities depicted in FIG. 1, certain data challenges in the clinical trial space are considered. In one example, longitudinal immutability of records is achieved because the tracking techniques make use of distributed ledger. Data integrity breaches can be thwarted by immutability of records. In another example, the present techniques automate processes using smart contracts. In a further example, audit and validation, including visibility for audit & compliance—FDA and other regulatory bodies, is provided.

In another aspect, the tracking systems and processes described herein include security, and provided that data in transit and at rest is encrypted. In an additional embodiment, identity management is handled cryptographically.

Advantageously, the present system achieves cost reduction from reduced errors and need for reconciliation since the technique provides a single source of truth that is shared by the entities 102-106 privy to the shared facts for a given transaction.

In another example, real-time reporting & updates, transaction processing, and data exchange are included. In a further example, the systems and methods provide permissioned access, e.g., so that only the participants involved know a transaction exists.

By way of example, blockchain and distributed ledger may be improved by providing application specific variations for the clinical trial space.

The term “distributed ledger” may use a “blockchain” in its implementation In one embodiment, a distributed ledge is a record of consensus between network participants with an audit trail of shared ledger entries that have been validated by the participants.

Returning now to FIG. 1, during clinical trials, human biological samples are collected at clinical investigator sites, such as at drug development entities 102, over an extended period. Flawless administration of the chain of custody is critical to sample management because correct handling impacts both the course and results of clinical trials.

Successful execution of clinical trials involves the coordination of a myriad of resources and processes. Maintaining visibility into the chain of custody can be challenging for many reasons, as can providing the level of data integrity and traceability required by regulatory authorities. At present, the chain of custody spans multiple stakeholders and disparate data systems, augmented by extensive processes at the clinical investigator sites. The result is excessive manual data entry, the need for coordination Patent Application between internal and external teams, frequent opportunities for error and difficult end-of-trial reconciliation.

Sample management can involve missing or contaminated samples, incomplete data, delays in reporting results, and substandard logistics. This leads to higher costs, lack of transparency, and other problems that can be avoided with the use of better technology like distributed ledger. Collectively, these factors represent a case for implementing distributed ledger:

The FDA and other regulatory bodies are tightening their scrutiny of traceability and Chain of Custody (CoC).

There is no industry standard regarding the exchange of CoC information, and conventional systems require that each vendor and participant in the CoC implements what they need. Assembling a full CoC record is a significant challenge, due to the various participants and disparate systems in use. Simply stated, clinical trial Sponsors benefit from full end-to-end visibility, as described herein.

Drug development entities 102 also must track other information in addition to sample management records, such as patient consent records and trial data records. Supply chain entities 104 have other information to track as well, including Inventory management, Regulatory compliance and Logistics compliance information. Distribution channels 106 also track Trade marketing, Returns authentication and Counterfeit prevention information.

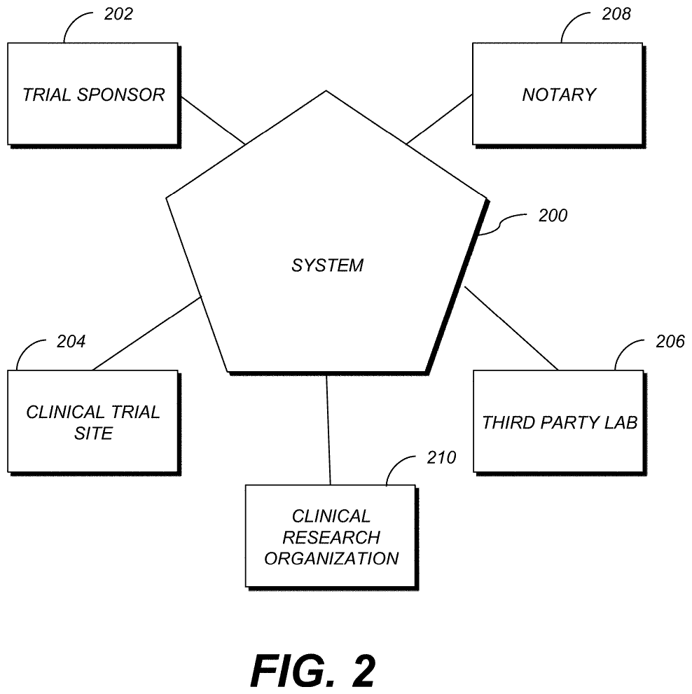

FIG. 2 depicts a block diagram of a system 200 for tracking clinical samples and trials. In the embodiment of FIG. 2, system 200 includes a digital ledger, e.g., implemented using a Digital Ledger Technology (DLT) software package, such as Corda DLT.

Note that a trust boundary exists between the system 200 and each entity 202-210 and that the system 200 includes smart contracts on distributed ledger that are used across the entities. Continuing, in the embodiment of FIG. 2, a pharma trial sponsor 202, clinical trial site 204, third party lab(s) 206, notary 208, and clinical research organization (CRO) 210 all have access to the system, including the distributed ledger functionality thereof. The distributed ledger of the system 200 enables data sharing with smart contracts.

For instance, a process flow is described as follows. Among the participants 202-208, system 200 ensures a streamlined and transparent process across network participants. In addition, system 200 can prove that real-time status updates can be tracked efficiently. Further, system 200 can achieve a scalable, transparent, and cost-effective process with a distributed ledger approach. Also, system 200 can allow for automation of receipt, reporting, and reconciliation, by standardizing and integrating the process. As another example, system 200 can utilize a workflow-based application so that users of each entity can visualize and track details in real time. As a further example, system 200 can utilize a node explorer application to provide visualization of the decentralized, distributed aspects of the distributed ledger network.

The sample tracking solution of system 200 can dramatically streamline the collection and sharing of information. Multiple stakeholders (e.g., participants 202-208), each with their own systems and data repositories, represent an intelligent distributed network for gathering and disseminating clinical and operational data relevant to the trial and its outcome. Each stakeholder can share data at a granular level, in real-time, and on a permissioned basis using smart contracts i.e. a subset of the data can be shared with one or more participants based on the configuration and logic in the Smart Contract. The Smart Contract is automatically triggered when pre-defined rules are met. Transactions are written to the ledger are digitally signed and timestamped, ensuring provenance and immutability of the record at a point in time.

Smart contracts can improve the efficiency and transparency of data management with respect to sample management. The need to reconcile between entities is eliminated and trial audit processes are expedited. Transparency and traceability of facts are important to the validity of clinical trials.

The benefits of this approach include: Assurance of data quality; Verifiability of provenance; Elimination of reconciliation between entities; Immutable audit history; Real-time data dissemination; A single source of truth.

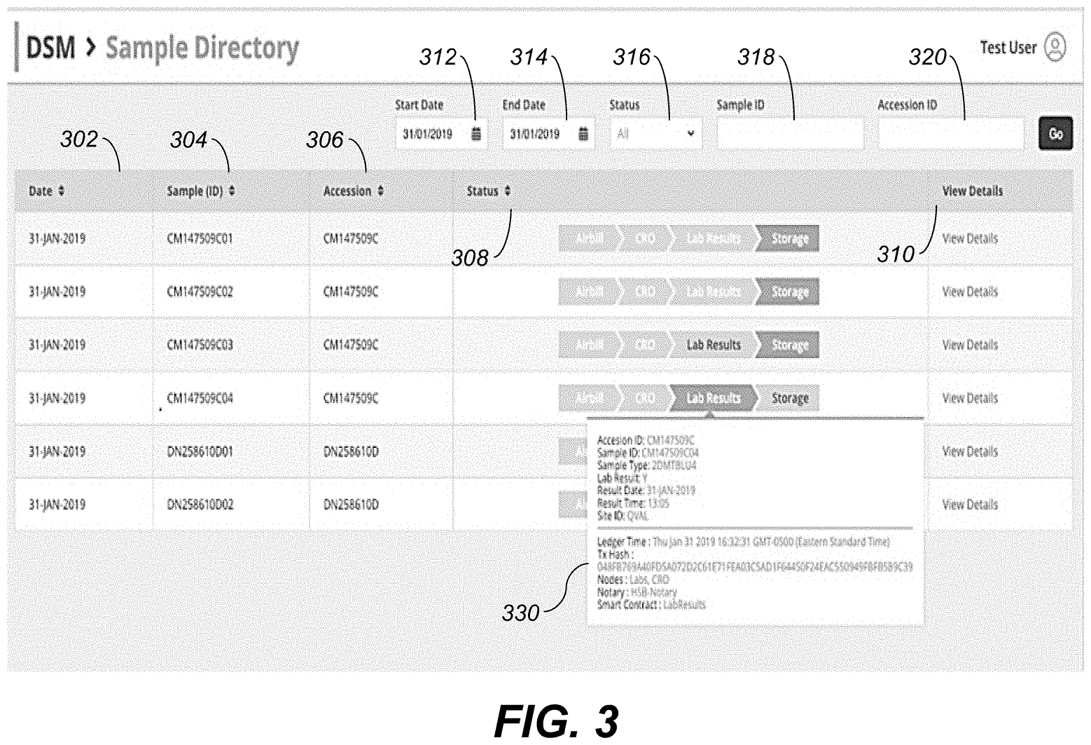

FIG. 3 depicts a graphical user interface of digital sample manager (DSM) application 300. In the example of FIG. 3, one of the graphical user interface screens of DSM application 300 allows an interface where stakeholder entities can easily locate details of the transactions in which they participate in. The application is deployed on each node and has an additional security layer where users, roles, and permissions can be administered. Data is pulled from both ON- and OFF-ledger databases to tie the distributed ledger-based information with the enterprise systems of the entities, providing actionable intelligence to end users.

By way of example, DSM application 300 allows data of the transaction as follows. The DSM application 300 displays for each transaction the date 302, sample ID 304, accession number 306, status 308, and details link 310. A set of filters 312-320 may be used to search transactions. The status 308 indicates where in the process each transaction stands, such as airbill, CRO, lab results, and storage.

Additionally, a Node Explorer application may be deployed, which is an administrative application for DevOps/MSO staff of the participating entities. Node Explorer helps with the granular details of the underlying data—entities see only their own information from the on-ledger data store.

Thus, unlike conventional systems, the present techniques are a practical application in the field of health care management and allow for multiple entities to participate in clinical trials and sample management without the need for trusting any one single entity, because the distributed ledger and/or blockchain features allow the entities to make their own determination that data is valid on the system. In addition, the role-specific nature of the specific smart contracts entered into by the parties allows for a structure by which the system may operate, rather than through the use of a plain blockchain without any defined roles.

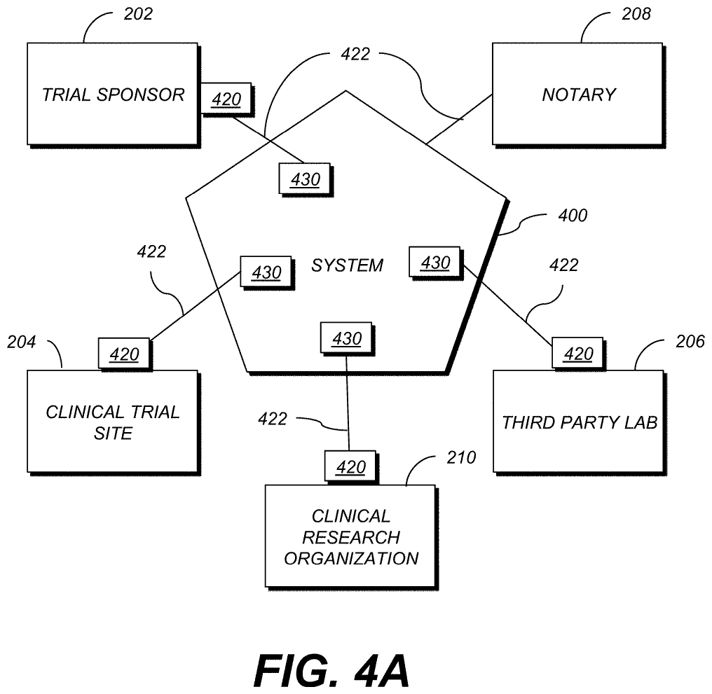

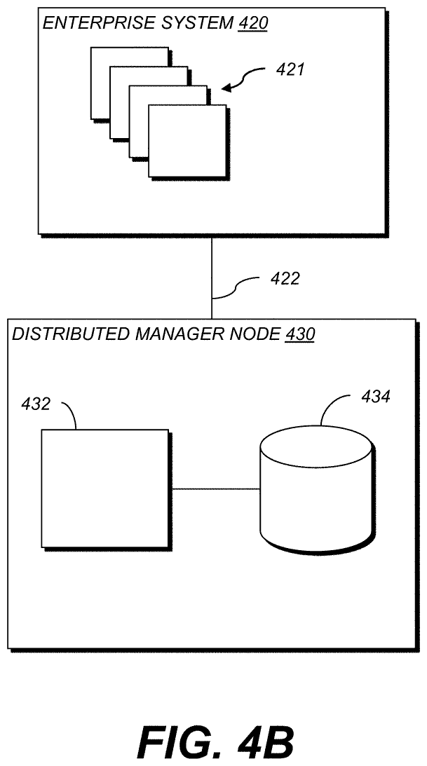

FIGS. 4A & 4B are block diagrams providing further implementation details of system 200, which includes multiple participants tracking clinical samples and trials. In the embodiment of FIG. 4A, legacy or existing enterprise systems 420, associated with each of the participants 202-208, are equipped with the ability to communicate via application programming interfaces (API) 422 to a distributed ledger node 430 to achieve a permissioned distributed de-centralized network. In this example, a cloud infrastructure may be used for implementation. In one specific example, cloud infrastructure may use Microsoft Azure cloud for implementation. In such a case, software code may be deployed and executed in the Microsoft Azure cloud utilizing Ds-v3 Virtual Instances with premium storage. Continuing with this example, the infrastructure can be created and managed as code with HashiCorp's Terraform to provide consistency and repeatability.

A working example of implementation details of one or more embodiments of the present disclosure is presented in FIG. 4B. In the embodiment of FIG. 4B, enterprise system 420 includes numerous enterprise servers 421. Distributed ledger node 430 can include a distributed ledger node 432 and a database 434. For example, distributed ledger node 430 can include Azure Instances running on Ubuntu 16.04LTS operating system, which has R3 Corda Open Source (Version 4.0), Microsoft SQL 2017 Express, Nginx, Node.js, and Java Runtime Engine. In such a case, smart Contract code may be written in Java. In addition, on Ledger data may be stored in a local SQL Express Instance on database 434. Next, an API may be exposed though node 430 that is utilized by both the node owner's existing Enterprise Systems as well as the DSM application via TLS. Such an API may be, for example, a Corda API. Further, the DSM application may run on Node.js. Further implementation details could include the use of Let's Encrypt for SSL/TLS certificates which are managed by Nginx and reverse proxied back to both the API (e.g., Corda API) and DSM ports.

By way of summary, FIGS. 4A & 4B disclose, in one or more embodiments, a system 400 for tracking clinical samples and clinical trials. For instance, the system 400 includes a distributed ledger for enforcing one or more smart contracts, a plurality of entities 202-210 joined to the distributed ledger, a plurality of nodes 430 corresponding to the plurality of entities, and a management application. The plurality of entities 202-210 include a trial sponsor entity 202, a trial site entity 204, a laboratory entity 206, and a research organization entity 210. Each entity of the plurality of entities comprises a corresponding enterprise system 420. The plurality of nodes 430 correspond to the plurality of entities 202-210. A corresponding node 430 of the plurality of nodes communicates with the corresponding enterprise system 420 of the plurality of entities using an applications programming interface (API) 422 to communicate with the distributed ledger. The plurality of entities 202-210 communicate using the API 422 to engage in one or more smart contracts managed by the distributed ledger on system 400 and related to the clinical samples and the clinical trials. A digital sample manager application provides a trusted user interface into the status of the one or more smart contracts.

In one specific example, each of the entities reads information and writes information from the distributed ledger. In such a case, the initiating entity, such as the sponsor, will initiate a study or clinical trial, and use a smart contract on the distributed ledger to initiate the parameters of the study. Each of the entities will engage with the sponsor on the distributed ledger to agree to perform a specific role in the study. The role will be specific to the individual entity. For example, the laboratory entity will perform tests, the clinical research organization will coordinate patient trials, and the trial site will conduct trials. In such an embodiment, the notary will be used to validate any data that is provided with respect to the trial by the entities, so that the data is validated on, e.g., a blockchain of the distributed ledger. Note that in some embodiments, many of each type of entity may be involved at least in the early stages, in bidding or offering services for a specific trial. For example, many laboratories, many CROs, many trial sites may offer to participate in the study by writing a response to the proposal on the distributed ledger, and the sponsor can then accept and engage in a smart contract with those entities.

In another example, the present system includes a distributed ledger based on a blockchain. Each entity writes their data to the blockchain so that the data cannot be changed. The different entities can have different views into different subsets of the data so that they do not see all the data. For example, a CRO can see data related to organizing a trial, but may not have access to specific patient data, for example, allowing for the system to comply to requirements to segregate data imposed by law. In another example, the data can be sequestered by the system so that trial results are secured.

In one embodiment, the one or more smart contracts comprise information of a chain of custody of samples and results of clinical trials, and a digital sample manager application enables each of the plurality of entities to access the information of the chain of custody of the samples and the results of clinical trials. In another embodiment, the one or more smart contracts comprise a smart contract initiated by the trial sponsor entity with the research organization entity to conduct and run tests, the smart contract being managed by the distributed ledger. In a further embodiment, the one or more smart contracts comprise a smart contract initiated by the trial sponsor entity with the trial site entity to identify patients and collect samples, the smart contract being managed by the distributed ledger.

In one implementation, the one or more smart contracts comprise a smart contract initiated by the trial sponsor entity with the laboratory entity to analyze samples, the smart contract being managed by the distributed ledger. In another implementation, the one or more smart contracts comprises at least one smart contract joined by the trial sponsor entity, the trial site entity, the laboratory entity and the research organization entity, wherein the at least one smart contract tracks data related to clinical samples and trials as the clinical samples and trials proceed through the plurality of entities. In a further implementation, a notary entity is included for validating transactions on the distributed ledger.

In one example, the one or more smart contracts govern one or more of a custody or a laboratory test of the clinical samples and the clinical trials. In another example, the distributed ledger operates on a cloud based platform. In a further example, the distributed ledger comprises a blockchain.

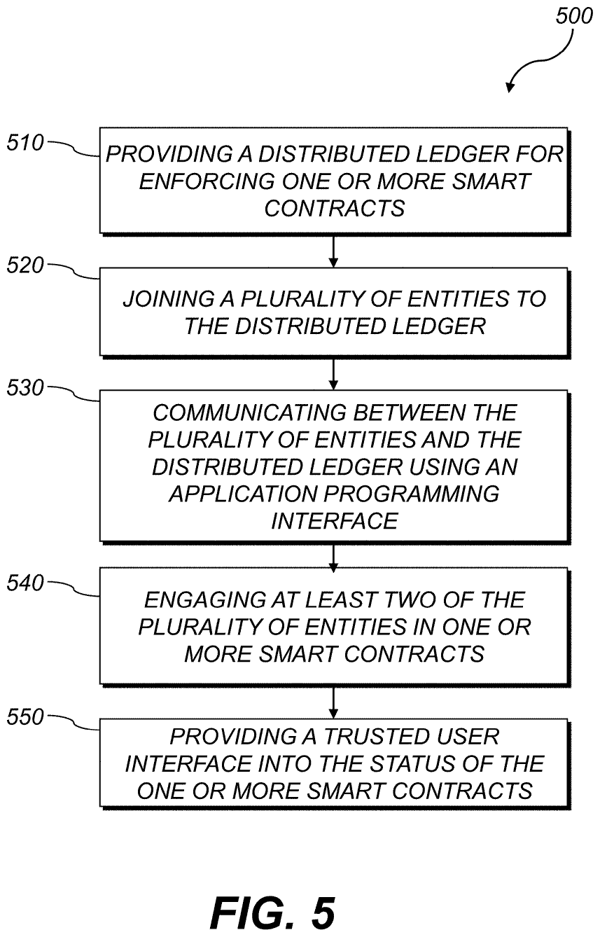

FIG. 5 is a flow chart of an exemplary method 500 of tracking clinical samples and trials. The method 500 at block 510 provides a distributed ledger for enforcing one or more smart contracts. As noted above, the distributed ledger includes, e.g., a blockchain.

The method 500 at block 520 joins a plurality of entities to the distributed ledger, the plurality of entities comprising a trial sponsor entity, a trial site entity, a laboratory entity, and a research organization entity.

In one example, the joining at block 520 comprises joining the trial sponsor entity, the trial site entity, the laboratory entity and the research organization entity in at least one smart contract, the at least one smart contract tracking data related to clinical samples and trials as the clinical samples and trials proceed through the plurality of entities. In another example, the joining at block 520 comprises joining a notary entity to the distributed ledger for validating transactions.

The method 500 at block 530 communicates between the plurality of entities and the distributed ledger using an application programming interface;

In one example, the communicating at block 530 comprises governing one or more of a custody or a laboratory test of the clinical samples and the clinical trials with a smart contract. In another example, the communicating at block 530 comprises deploying the distributed ledger on a cloud based platform.

The method 500 at block 540 engages at least two of the plurality of entities in one or more smart contracts managed by the distributed ledger and related to the clinical samples and the clinical trials.

In one example, the engaging at block 540 comprises initiating a smart contract between the trial sponsor entity and the research organization entity to conduct and run tests, and managing the smart contract using the distributed ledger. In another example, the engaging at block 540 comprises initiating a smart contract between the trial sponsor entity and the trial site entity to identify patients and collect samples, and managing the smart contract using the distributed ledger. In a further example, the engaging at block 540 comprises initiating a smart contract between the trial sponsor entity and the laboratory entity to analyze samples, and managing the smart contract using the distributed ledger.

In a specific implementation, the one or more smart contracts comprise information of a chain of custody of samples and results of clinical trials, and the method further comprises enabling each of the plurality of entities to access the information of the chain of custody of the samples and the results of clinical trials.

The method 500 at block 550 providing a trusted user interface into the status of the one or more smart contracts.

To the extent that the claims recite the phrase “at least one of” in reference to a plurality of elements, this is intended to mean at least one or more of the listed elements, and is not limited to at least one of each element. For example, “at least one of an element A, element B, and element C,” is intended to indicate element A alone, or element B alone, or element C alone, or any combination thereof “At least one of element A, element B, and element C” is not intended to be limited to at least one of an element A, at least one of an element B, and at least one of an element C.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

What is claimed is:

1. A system for tracking clinical samples and clinical trials, the system comprising:
a distributed ledger for enforcing one or more smart contracts;
a plurality of entities joined to the distributed ledger, the plurality of entities comprising
a trial sponsor entity,
a trial site entity,
a laboratory entity, and
a research organization entity;
a plurality of enterprise systems, wherein each entity of the plurality of entities comprises a corresponding enterprise system of the plurality of enterprise systems;
a plurality of nodes corresponding to the plurality of entities, wherein a corresponding node of the plurality of nodes communicates with the corresponding enterprise system of the plurality of entities using an applications programming interface (API) to communicate with the distributed ledger; and
a management application, wherein the plurality of entities communicate using the API to engage in one or more smart contracts managed by the distributed ledger and related to the clinical samples and the clinical trials, and wherein the management application provides a trusted user interface into the status of the one or more smart contracts.

2. The system of claim 1, wherein the one or more smart contracts comprise information of a chain of custody of samples and results of clinical trials, and the digital sample manager application enables each of the plurality of entities to access the information of the chain of custody of the samples and the results of clinical trials.

3. The system of claim 1, wherein the one or more smart contracts comprise a smart contract initiated by the trial sponsor entity with the research organization entity to conduct and run tests, the smart contract being managed by the distributed ledger.

4. The system of claim 1, wherein the one or more smart contracts comprise a smart contract initiated by the trial sponsor entity with the trial site entity to identify patients and collect samples, the smart contract being managed by the distributed ledger.

5. The system of claim 1, wherein the one or more smart contracts comprise a smart contract initiated by the trial sponsor entity with the laboratory entity to analyze samples, the smart contract being managed by the distributed ledger.

6. The system of claim 1, wherein the one or more smart contracts comprises at least one smart contract joined by the trial sponsor entity, the trial site entity, the laboratory entity and the research organization entity, wherein the at least one smart contract tracks data related to clinical samples and trials as the clinical samples and trials proceed through the plurality of entities.

7. The system of claim 1, further comprising a notary entity for validating transactions on the distributed ledger.

8. The system of claim 1, wherein the one or more smart contracts govern one or more of a custody or a laboratory test of the clinical samples and the clinical trials.

9. The system of claim 1, wherein the distributed ledger operates on a cloud based platform.

10. The system of claim 1, wherein the distributed ledger comprises a blockchain.

11. A method for tracking clinical samples and clinical trials, the method comprising:
providing a distributed ledger for enforcing one or more smart contracts;
joining a plurality of entities to the distributed ledger, the plurality of entities comprising a trial sponsor entity, a trial site entity, a laboratory entity, and a research organization entity;
communicating between the plurality of entities and the distributed ledger using an application programming interface;
engaging at least two of the plurality of entities in one or more smart contracts managed by the distributed ledger and related to the clinical samples and the clinical trials; and
providing a trusted user interface into the status of the one or more smart contracts.

12. The method of claim 11, wherein the one or more smart contracts comprise information of a chain of custody of samples and results of clinical trials, and the method further comprises enabling each of the plurality of entities to access the information of the chain of custody of the samples and the results of clinical trials.

13. The method of claim 11, wherein the engaging comprises initiating a smart contract between the trial sponsor entity and the research organization entity to conduct and run tests, and managing the smart contract using the distributed ledger.

14. The method of claim 11, wherein the engaging comprises initiating a smart contract between the trial sponsor entity and the trial site entity to identify patients and collect samples, and managing the smart contract using the distributed ledger.

15. The method of claim 11, wherein the engaging comprises initiating a smart contract between the trial sponsor entity and the laboratory entity to analyze samples, and managing the smart contract using the distributed ledger.

16. The method of claim 11, wherein the joining comprises joining the trial sponsor entity, the trial site entity, the laboratory entity and the research organization entity in at least one smart contract, the at least one smart contract tracking data related to clinical samples and trials as the clinical samples and trials proceed through the plurality of entities.

17. The method of claim 11, wherein the joining comprises joining a notary entity to the distributed ledger for validating transactions.

18. The method of claim 11, wherein the communicating comprises governing one or more of a custody or a laboratory test of the clinical samples and the clinical trials with a smart contract.

19. The method of claim 11, wherein the communicating comprises deploying the distributed ledger on a cloud based platform.

20. The method of claim 11, wherein the distributed ledger comprises a blockchain.
